(19) United States
(12) Patent Application Publication
(10) Pub. No.: US 2003/0133895 A1
(43) Pub. Date: Jul. 17, 2003

(54) Pressed cosmetic powder and process for making

(75) Inventors: Robert H. China, Midland Park, NJ (US); Harold E. Pahlck, Waldwick, NJ (US); Arvind Shah, Suffern, NY (US)

Correspondence Address: Charles N.J. Ruggierio, Esq., Ohlandt, Greeley, Ruggerio & Perle, L.L.P., 10th Floor, One Landmark Square, Stamfort, CT, 06901-2682, US

(73) Assignee: AVON PRODUCTS, INC., NEW YORK, NY

(21) Appl. No.: 10/032,787

(22) Filed: Dec. 26, 2001

(51) Int. Cl. A61K 7/021; A61K 7/06

(52) U.S. Cl. 424/70.12

(57) ABSTRACT

The present invention provides a cosmetic powder composition that has superior aesthetic properties over the prior art. The present invention is preferably a pressed powder cosmetic composition that includes silicone elastomer particles that have a JISA hardness index less than about 45. The cosmetic powder cosmetic compositions of the present invention have a hardness value less than about 1500 grams and/or a percent recovery value greater than about 25.

### BACKGROUND OF THE INVENTION

1. Field of the Invention

The present invention relates to pressed powder cosmetic compositions. More particularly, the present invention relates to pressed powder cosmetic compositions having silicone elastomer particulates of a defined hardness index. Still more particularly, the present invention relates to pressed powder cosmetic compositions having superior binding properties. Further still, the present invention relates to pressed powder cosmetic compositions having superior aesthetics, such as springiness when touched, yet is commercially viable, e.g., passes the “drop test”. In addition, the present invention relates to a process for preparing such compositions.

2. Description of the Prior Art

Cosmetic powders have been commercially available in both loose and pressed powder product forms. Loose powders have the advantage of easy application but suffer from poor portability (prone to product spillage). Pressed powders have the consumer advantages of portability (less prone to product spillage) and hygiene (less likelihood of inhaling powder particles), but are hard to the touch and, thus, less easy to apply. Pressed powders have been the predominant commercial product form.

Representative of the pressed cosmetic powder art are published applications JP 11-216836, JP 10-233084, JP 07-138555, JP 06-085739, and JP 06-071493. These published applications provide cosmetic powders having particles of an organopolysiloxane elastomer.

It would be desirable to have a cosmetic composition in the form of a compressed, dry powder with improved aesthetic appearance, superior binding properties, and a certain degree of springiness for ease of application. However, until now there was not a pressed powder that provided these desired characteristics, especially such a powder that could be manufactured, shipped and sold in commercial quantities.

### SUMMARY OF THE INVENTION

It is an object of the present invention to provide a pressed powder cosmetic composition that exhibits superior binding properties and springiness.

It is another object of the present invention to provide a pressed powder cosmetic composition that has an excellent aesthetic appearance and/or feel.

It is a further object of the present invention to provide a process of making a pressed powder cosmetic composition that has superior binding properties and springiness, and provides an excellent aesthetic appearance and/or feel.

These and other objects and advantages of the present invention are achieved by incorporating one or more silicone elastomers having a JISA hardness index less than about 45, preferably about 2 to less than about 45, and more preferably about 4 to about 35. Optimally, the JISA hardness index of the silicone elastomers is about 20 to about 35. The foregoing indexes are measured according to the Japanese Industrial Standards JIS K 6253 method (published by Japanese Industrial Standards Association). The present invention also includes the process of making such pressed powder cosmetic compositions.

### DESCRIPTION OF THE DRAWINGS

FIG. 1 is bar graph illustrating the percent recovery (springiness) of four examples of the present invention relative to a sponge and a prior art pressed powder (control).

FIG. 2 is a bar graph illustrating the hardness of the four examples of the present invention relative to the sponge and the prior art pressed powder (control).

FIG. 3 is bar graph illustrating the percent recovery (springiness) of ten additional examples of the present invention relative to the sponge and the prior art pressed powder (control).

FIG. 4 is a bar graph illustrating the hardness of the ten additional examples of the present invention relative to the sponge and the prior art pressed powder (control).

FIG. 5 is a graphical representation of the Hardness cycle 1 and Area cycle 1 (depicted by curve A) and Hardness cycle 2 and Area cycle 2 (depicted by curve A′) for Ex. 7 of the present invention.

### DESCRIPTION OF THE INVENTION

It was surprising and unexpected that a pressed powder composition having an excellent aesthetic appearance and exhibiting superior binding and springiness could be obtained. Further surprisingly, the composition has desirable aesthetics such as softness to the touch, smooth feel upon application, velvety appearance and a satiny finish. Still further, the composition exhibits excellent adherence and sebum/oil absorption capability. These characteristics, when taken alone or collectively, represent a clear advance in pressed powder technology. These advantages were achieved by the pressed powder composition of the present invention, preferably made by the process of the present invention.

As used herein, “a pressed powder composition” is a composition of dry, free-flowing powder that has been pressed to a reduced volume and to a form such that, for example, when pressed into a rigid package, it will substantially maintain its shape and consistency regardless of the positioning of the package.

The pressed powder composition of the present invention includes a dry powder phase and a liquid binder phase. The liquid binder phase provides adhesion for particulates of the dry powder phase so that a pressed powder composition will form and hold. The dry powder phase has about 1 weight percentages (wt %) to about 99 wt %, more preferably about 82 wt % to about 98 wt %, most preferably from about 88 wt % to about 92 wt %, of the total weight of the pressed powder composition.

The dry powder phase includes particulates or particles of one or more silicone elastomers. The silicone elastomer particulate(s) have a JISA hardness index of less than about 45, preferably about 2 to less than about 45, more preferably about 4 to about 35, and optimally about 20 to about 35. The foregoing indexes are measured according to the Japanese Industrial Standards JIS K 6253 method (published by Japanese Industrial Standards Association). Furthermore, the silicone elastomer particulates are present in an amount about 1 wt % to about 25 wt % of the total weight of the composition. A preferred amount is about 3 wt % to about 15 wt % and a most preferred amount is about 3 wt % to about 12 wt %, of the total weight of the composition.

Examples of the preferred silicone elastomers are Gransil EPS and Gransil EP-LS from Grant Industries, Inc.; Dow Corning 9505 (formerly, Trefil E-505C) and Dow Corning 9506 (formerly, Trefil E-506C) from Dow Corning, Corp; and Shin Etsu X-52-875 from Shin Etsu. The Grant Industries products are known by the INCI name of polysilicone-11 and laureth-12, while the Dow Corning products are known by the INCI name of dimethicone/vinyl dimethicone crosspolymer. Dow Corning 9506 and Shin Etsu X-52-875 are preferred.

The dry powder phase also includes other particulate ingredients, such as a filler. Such fillers that can be used in the dry powder phase include mineral silicate, starch, kaolin, nylon, zinc oxide, titanium oxide, precipitated calcium carbonate, synthetic polymer powder, as well as other fillers known in the art, or any combinations thereof. The fillers may have hydrophobic or hydrophilic surfaces. Hydrophobicity may be imparted via treatment with a silicone, such as a methicone or dimethicone, or a polymer such as Fomblin®.

The most preferred fillers are mineral silicates, such as mica and talc. When the mineral silicate is mica, it is preferably provided in the form of flakes. Each flake has a size ranging from about 2 μm to about 200 μm, and preferably from about 5 μm to about 70 μm. The thickness of each flake ranges from about 0.1 μm to about 5 μm, and more preferably from about 0.2 μm to about 3 μm. The mica can be of natural origin (for example, muscovite, margarite, rescolithe, lipidolithe, biotite), or synthetic origin. Preferably, the mica is substantially transparent and imparts to the skin a satin-like appearance. In the cosmetic compositions of the present invention, mica filler is present in an amount preferably about 0.1 wt % to about 70 wt %, more preferably about 0.1 wt % to about 50 wt %, and most preferably about 1 wt % to about 10 wt %, based on the total weight of the composition.

When the filler includes talc, the talc may be present in an amount about 0.1 wt % to about 99 wt %, preferably about 10 wt % to about 50 wt %, and more preferably about 20 wt % to about 35 wt %, of the total weight of the composition. Preferably, the average particle size of the talc filler should range from about 0.5 microns to about 9 microns, and more preferably from about 4 microns to about 8 microns.

Other preferred mineral silicates that can be used in the present invention are phyllosilicates and tectosilicates including, for example, pyrophyllite, chlorite, chrysotile, antigorite, lizardite, kaolinite, dickite, nacrite, halloysite, montmorillonite, nontronite, saponite, sauconite, and bentonite; natrolites such as natrolite, nesolite, scolecite, and thomsonite; heulandites such as heulandite, stilbite, epistibite; and zeolites such as analcite, harmotone, phillipsite, chabazite, gmelinite, or any combinations thereof.

Another preferred filler is a synthetic polymer powder. Such powders include polyethylene, polyesters (for example, polyethylene isophthalate or terephthalate), N-lauryl lysine, polyamides (for example, nylon), or any combinations thereof. The particles of these powders typically have a size of less than about 50 μm. Also, the particles possess feel-modifying/rolling/slip properties that impart to the skin a velvety feel. Preferably, the synthetic polymer powder filler is present in an amount about 1 wt % to about 40 wt %, and more preferably about 5 wt % to about 25 wt %, based on the total weight of the composition.

The present compositions may also have a metallic soap to impart an unctuous feel and facilitate the adherence of the composition to the skin. Metallic soaps may be derived from one or more organic carboxylic acids having 8 to 22 carbon atoms. Preferably, the organic carboxylic acid has 12 to 18 carbon atoms. Useful examples of such metallic soaps include zinc stearate, magnesium stearate, lithium stearate, zinc laurate, magnesium myristate, or any combinations thereof. These soaps are present generally in the form of particles having a size less than 10 μm. The metallic soaps are preferably present in an amount about 1 wt % to about 10 wt %, and more preferably about 2 wt % to about 7.5 wt %, based on the total weight of the composition.

The present compositions may also have a pigment, such as mineral and/or organic pigments. Although the present invention can accommodate variations in amount of pigment to provide a desired shade, the pigment is typically present from about 0.1 wt % to about 15 wt % based on the total weight of the composition.

Representative mineral pigments include, for example, titanium dioxide (rutile or anatase) optionally surface treated and listed in the Color Index under reference CI 77891; black, yellow, red and brown iron oxides listed in Color Index under references CI 77499, 77492 and 77491; managanese violet (CI 77742); ultramarine blue (CI 77007); chromium oxide (CI 77288); hydrated chrominum oxide (CI 77289); ferric blue (CI 77510), or any combinations thereof.

Other exemplary pigments are white nacreous pigments, such as mica covered with titanium oxide, bismuth oxychloride; and colored nacreous pigments, such as titanium mica with iron oxides, titanium mica with ferric blue or chromium oxide, titanium mica with an organic pigment of the aforementioned type, as well as those based on bismuth oxychloride.

Additional exemplary pigments include, for example, the following: D&C Red No. 19 (CI 45170); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); D&C Red No. 19 (CI 73360); D&C Red No. 19 (CI 45430); and carmine lakes (CI 75470); FD&C Red #40 (CI# 16035); FD&C Blue #1 (CI# 42090); FD&C Yellow #5 (CI# 19140); or any combinations thereof.

The dry powder phase may optionally have an inorganic salt. Inorganic salts provide binding properties with less glazing in the final pressed powder composition. Inorganic salts that can be used in the compositions of the present invention include those inorganic salts listed at pages 829 through 830 of the Third Edition of the International Cosmetic Ingredient Handbook (1995), which is incorporated herein by reference. The preferred salts for use in the present compositions include calcium carbonate, calcium chloride, calcium phosphate, calcium silicate, calcium sulfate, or any combinations thereof.

The present composition may optionally have a synthetic polymer powders (including salts) which provides a nice “payoff” and a silky, luxurious feel on the skin. Such synthetic polymers include those listed at pages 850 through 852, except acrylic acid/acrylonite copolymer, adipic acid/CHDM/MA/Neopentyl Glycol/trimellitic anhydride copolymer, adipic acid/diethylene glycol/glycerin crosspolymer, adipic acid/diethylenetriamine copolymer, adipic acid/dimethylaminohydroxypropyl diethylenetriamine copolymer, adipic acid/epoxypropyl diethylenetriamine copolymer, adipic acid/fumaric acid/phthalic acid/twistanedimethanol copolymer, adipic acid/isophthalic acid/neopentyl glycol/trimethylolpropane copolymer, allyl stearate/VA copolymer, aminoethylacrylate phosphate/acrylates copolymer, aminopropyl dimethicone, ammonium acrylates/acrylonitrogens copolymer, ammonium acrylates copolymer, ammonium polyacrylate, ammonium styrene/acrylates copolymer of the Third Edition of the International Cosmetic Ingredient Handbook (1995), which is incorporated herein by reference. Preferably, the synthetic polymers include acrylamide/ammonium acrylate copolymer, acrylamides/acrylates/DMAPA/methoxy PEG methacrylate copolymer, acrylamides copolymer, acrylamide/sodium acrylate copolymer, acrylamidopropyltrimonium chloride/acrylamide copolymer, acrylamidopropyltrimonium choloride/acrylates copolymer, acrylates/acetoacetoxyethyl methacrylate copolymer, acrylates/acetoacetoxyethyl methacrylate copolymer, acrylates/acrylamide copolymer, acrylates/ammonium methacrylate copolymer, acrylates copolymer, acrylates/hydroxyesters acrylates copolymer, acrylates/octylacrylamide copolymer, acrylates/PVP copolymer, acrylates/steareth-50 acrylate copolymer, acrylates/steareth-20 methacrylate copolymer, acrylates/VA copolymer, acrylates/VA crosspolymer, acrylates/vinyl isodecanoate crosspolymer, ethyllene/acrylic acid copolymer, ethylene/acrylic acid/VA copolymer, ethylene/MA copolymer, ethylene/VA copolymer, nylon-6, nylon-11, nylon-12, nylon-66, methacrylate copolymer, polymethyl methacrylate, or any combinations thereof.

The pressed powder composition of the present invention also includes a liquid binder phase. The term “liquid binder phase” means binder phases that are liquid at room temperature, or at any point in the manufacturing process. The liquid binder phase may include, among others, oils, hydrocarbons, liquid synthetic esters, silicone oils, waxes, or silicone emulsifiers. The liquid binder phase comprises about 1 wt % to about 20 wt %, more preferably about 2 wt % to about 18 wt %, and most preferably from about 8 wt % to about 12 wt %, of the total weight of the composition.

The liquid binder phase may have one or more surfactants and emulsifying agents to provide uniformity to the liquid binder phase and/or to maximize pigment color performance. Surfactants and emulsifying agents that may be used in the present compositions include those listed at pages 919 and 923, except PEG-9 stearate through poloxamine 1504 and polysorbate 20 through PPG-20-buteth-30 of the Third Edition of the International Cosmetic Ingredient Handbook (1995), which is incorporated herein by reference. The preferred surfactants/emulsifying agents are cetyl dimethicone copolyol, cetyl glyceryl ether/glycerin copolymer, polygylceryl-3 diisostearate, or any combinations thereof.

The liquid binder phase may also include silicones and silanes. Silicones and silanes are feel-modifiers. They improve slip, improve wear and provide moisturization benefits. Silicones and silanes that can be used in the present compositions include those listed at pages 844 through 845 of the Third Edition of the International Cosmetic Ingredient Handbook (1995), which is incorporated herein by reference. The preferred silicones/silanes are dimethicone, trimethylsiloxysilicate, or any combination thereof. The most preferred silicone/silane is DOW CORNING 593 FLUID.

The liquid binder phase may also have an ester as a cosolubilizer and/or cosolvent to assist in dissolving solid resins and/or elastomers. Esters that may be used in the present compositions include those listed at pages 813 through 818 of the Third Edition of the International Cosmetic Ingredient Handbook (1995), which is incorporated herein by reference. The preferred esters are pentaerythrityl dioleate, pentaerythrityl distearate, pentaerythrityl hydrogenated rosinate, pentaerythrityl isostearate/caprate/caprylate/adipate, pentaerythrityl rosinate, pentaerythrityl stearate, pentaerythritylstearate/caprate/caprylate adipate, pentaerythrityl tetraabietate, pentaerythrityl tetraacetate, pentaerythrityl tetrabehenate, pentaerythrityl tetrabenzoate, pentaerythrityl tetracaprylate/tetracaprate, pentaerythrityl tetracocoate, pentaerythrityl tetraisononanoate, pentaerythrityl tetraisostearate, pentaerythrityl tetralaurate, pentaerythrityl tetramyristate, pentaerythrityl tetraoleate, pentaerythrityl tetraperlargonate, pentaerythrityl tetrastearate, pentaerythrityl trioleate, or any combinations thereof. Pentaerythrityl tetraoctanoate is a most preferred ester.

The liquid binder phase may also include a hydrocarbon, preferably as an emollient and/or conditioning agent. Hydrocarbons that may be used in the present compositions include those listed at page 827, except azulene, butane, C10-13 alkane, C14-17 alkane, coal tar cyclohexane, p-cymene, deodorized kerosene, didecene, dicetylcyclohexane, dipentene, diphenyl-methane, ethane, gualazulene, heptane, hexane, isobutane, mineral spirits, mixed terpened, nitromethane, pentane, pristane, propane, red-petrolatum, toluene, turpentine and xylene of the Third Edition of the International Cosmetic Ingredient Handbook (1995), which is incorporated herein by reference, or any combinations thereof. Squalane is a preferred hydrocarbon.

The pressed powder compositions of the present invention can be used to deliver any type of cosmetic ingredient, e.g., fragrances, preservatives, and biological additives to provide skin benefits or treatment benefits. Useful biological additives include those listed at pages 865 through 870 of the Third Edition of the International Cosmetic Ingredient Handbook (1995), which is incorporated herein by reference. Moreover, by using the pigments and/or colorants discussed above, compositions of the present invention can be formulated to produce a variety of color cosmetic compositions suitable for use on the face, lips, eyes and body. As can be determined by those in the art, any optional ingredient/additive should be added to the phase, either dry powder phase or oil binder phase, with which it is most compatible.

The present invention is prepared by the following steps. The dry powder phase and the liquid binder phase are prepared separately. All powder particulate ingredients, including the silicone elastomer particulates and all fillers, are mixed to form a dry powder phase. Preferably, the dry powder phase is mixed until uniform. Suitable mixers/blenders are available and are known by those skilled in the art. The powder composition may also be jet milled by techniques and procedures known in the art.

In a separate vessel, the liquid binder phase is prepared either simultaneously, before or after the preparation of the dry powder phase. The liquid binder phase ingredients are mixed, preferably at a temperature about 75 to about 80 degrees F when the ingredients in the liquid binder phase are liquid at room temperature, but preferably the liquid binder phase is indexed at a temperature of about 175 to about 180 degrees F when the liquid binder phase includes solid ingredients, such as wax.

The liquid binder phase is then added slowly to the dry powder phase. Both phases are mixed, preferably in an Oysterizer™ at a high speed, until blended. The blended mixture is then preferably sifted through a sieve. One example of a suitable sieve is USA standard testing sieve/screen no. 35 having an opening number 020 (i.e., 550 micrometer/0.0197 inches).

After the liquid and powder phases have been blended and, preferably, sifted, pressing occurs in the range about 800 psi (gauge pressure) to about 2500 psi. Preferably, pressing of the mixture of ingredients takes place in the range about 1000 psi to about 1700 psi. The pressure ranges result in the formation of a pressed composition having excellent aesthetic appearance, superior binding, and superior sponginess/springiness.

### EXAMPLES

The objective was to compare hardness values and percent recovery as a way to determine the relative cushioning/springiness demonstrated by pressed cosmetic powders of the present invention as compared to a positive control (a sponge) and a negative control (a pressed powder without silicone elastomer particles, i.e., prior art).

The sponge applicator used as a reference had a height equal to the depth of the pan used for pressing the powders (i.e., 4 mm). Control samples were prior art cosmetic powders, i.e., without silicone elastomer particles.

Examples 1 through 4

| INGREDIENT | CONTROL | Ex. 1 | Ex. 2 | Ex. 3 | Ex. 4 |
|---|---|---|---|---|---|
| Dry Powder Phase | | | | | |
| Fillers (Sericite- | 86.73 | 77.73 | 72.275 | 77.73 | 72.275 |
| Silicone, Sericite | | | | | |
| FSE, Talc-Silicone | | | | | |
| Treated) | | | | | |
| Silk Powder | 0.200 | 0.200 | 0.167 | 0.200 | 0.167 |
| Preservatives | 0.300 | 0.300 | 0.250 | 0.300 | 0.250 |
| (Methylparaben) | | | | | |
| Pigments (Yellow (w/ | 0.62 | 0.62 | 0.516 | 0.62 | 0.516 |
| 20% talc extender), | | | | | |
| Iron Oxide Red, Brown | | | | | |
| (w/20% talc | | | | | |
| extender) and Black | | | | | |
| (w/20% talc | | | | | |
| extender) | | | | | |
| DOW-9505 | — | 9.000 | 16.667 | — | — |
| DOW-9506 | — | — | — | 9.000 | 16.667 |
| Liquid Binder Phase | | | | | |
| Squalane, | 12.15 | 12.15 | 10.125 | 12.15 | 10.125 |
| Pentaerythritol | | | | | |
| Tetraocanoate, | | | | | |
| Dimethyl/Trimethyl | | | | | |
| Polysiloxane and | | | | | |
| Fragrance | | | | | |
| TOTAL WT % | 100.000 | 100.000 | 100.000 | 100.000 | 100.000 |

Examples 5 through 14

| | Ex. | Ex. | Ex. | Ex. | Ex. | Ex. | Ex. | Ex. | Ex. | Ex. |
|---|---|---|---|---|---|---|---|---|---|---|
| Ingredients | 5 | 6 | 7 | 8 | 9 | 10 | 11 | 12 | 13 | 14 |
| DRY POWDER | | | | | | | | | | |
| PHASE | | | | | | | | | | |
| Fillers (Talc, | 93.4 | 83.8 | 77.8 | 61.2 | 93.4 | 77.8 | 61.2 | 93.4 | 77.8 | 61.2 |
| Mica, Hydrous | | | | | | | | | | |
| Calcium Silicate, | | | | | | | | | | |
| Nylon Powder, | | | | | | | | | | |
| Spherical | | | | | | | | | | |
| Polymethyl | | | | | | | | | | |
| Methacrylate, | | | | | | | | | | |
| Zinc Stearate, | | | | | | | | | | |
| Polyethylene, | | | | | | | | | | |
| Acrylate | | | | | | | | | | |
| Copolymer, | | | | | | | | | | |
| Fumed | | | | | | | | | | |
| Silica) | | | | | | | | | | |
| Preservatives | 0.7 | 0.7 | 0.7 | 0.7 | 0.7 | 0.7 | 0.7 | 0.7 | 0.7 | 0.7 |
| (Methyl Paraben, | | | | | | | | | | |
| Butyl Paraben, | | | | | | | | | | |
| Imidazolidinyl | | | | | | | | | | |
| Urea) | | | | | | | | | | |
| Pigments (Iron | 2.3 | 2.3 | 2.3 | 2.3 | 2.3 | 2.3 | 2.3 | 2.3 | 2.3 | 2.3 |
| Oxides) | | | | | | | | | | |
| Silicone | 1.0 | 4.0 | 10.0 | 20.0 | — | — | — | — | — | — |
| Elastomer | | | | | | | | | | |
| Particles | | | | | | | | | | |
| (Dow Corning | | | | | | | | | | |
| 9506) | | | | | | | | | | |
| Silicone | — | — | — | — | 1.0 | 10.0 | 20.0 | — | — | — |
| Elastomer | | | | | | | | | | |
| Particles | | | | | | | | | | |
| (Gransil Eps) | | | | | | | | | | |
| Silicone | — | — | — | — | — | — | — | 1.0 | 10.0 | 20.0 |
| Elastomer | | | | | | | | | | |
| Particles | | | | | | | | | | |
| (Shinetsu X-52- | | | | | | | | | | |
| 875) | | | | | | | | | | |
| Liquid Binder | | | | | | | | | | |
| Phase | | | | | | | | | | |
| Silicone Fluids | 1.0 | 4.4 | 4.4 | 7.8 | 1.0 | 4.4 | 7.8 | 1.0 | 4.4 | 7.8 |
| (Dimethicone 50 | | | | | | | | | | |
| Cst, Silicone | | | | | | | | | | |
| Fluid Sf-96-5) | | | | | | | | | | |
| Silicone Resin | 0.50 | 2.20 | 2.20 | 3.9 | 0.5 | 2.2 | 3.9 | 0.5 | 2.2 | 3.9 |
| (Dimethyl/ | | | | | | | | | | |
| Trimethyl | | | | | | | | | | |
| Polysiloxane) | | | | | | | | | | |
| Wax (Parrafin | 0.50 | 1.55 | 1.55 | 2.60 | 0.5 | 1.55 | 2.6 | 0.5 | 1.55 | 2.6 |
| Wax) | | | | | | | | | | |
| Emulsifier (Cetyl | 0.60 | 1.05 | 1.05 | 1.50 | 0.6 | 1.05 | 1.5 | 0.6 | 1.05 | 1.5 |
| Dimethicone | | | | | | | | | | |
| Copolyol) | | | | | | | | | | |
| TOTAL WT % | 100 | 100 | 100 | 100 | 100 | 100 | 100 | 100 | 100 | 100 |

The compositions were pressed into cosmetic pans at about 900 to about 1000 psi.

Measurement Protocol

The compositions were tested according to the following methodology. The test equipment was a STEVENS LFRA Texture Analyzer Model QTS-25. The software was a QTS-25 Software 10 Package.

QTS 25 Settings:

| | |
|---|---|
| MODE | Measure force in compression |
| TEST TYPE | TPA |
| TOTAL CYCLES | 2 |
| HOLD TIME | 0 |
| RECOVERY TIME | 0 |
| TRIGGER | 0.5 gm |
| TEST SPEED | 10 mm/min |
| RETURN SPEED | 10 mm/min |
| TARGET UNIT | Distance |
| DISPLAY UNITS | Gm |
| TARGET VALUE | 1 mm |
| TEST PROBE | 4 mm diameter, s/s (Ref. TA-24) |

Once positioned, the probe was centrally located above the surface of the sample and driven 1 mm into its surface. The 4 mm diameter probe traveled into the sample's surface until a trigger force of 0.5 gm was recorded. The forces generated increased until a peak force was attained at the target distance of 1 mm of penetration. The various peaks and troughs generated within the force deformation curve are indicative of the presence of trapped air or “grainy” texture encountered while penetrating to the target distance. The extent of the load (in grams) required to break or deform the surface is indicative of the sample hardness.

All parameters (i.e., Hardness cycle 1, Hardness cycle 2, Area cycle 1, and Area cycle 2) were automatically calculated within the instrumentation's software.

Result Definitions:

Hardness cycle 1 (gm): Peak Positive force generated within the 1^{st} compression cycle.

Positive Area cycle 1: Work applied to attain deformation within the compression cycle (e.g., Consistency).

Hardness cycle 2 (gm): Peak Positive force generated within the 2^{nd} compression cycle.

Positive Area cycle 2: Work applied to attain deformation within the 2^{nd} compression cycle (e.g., Springiness).

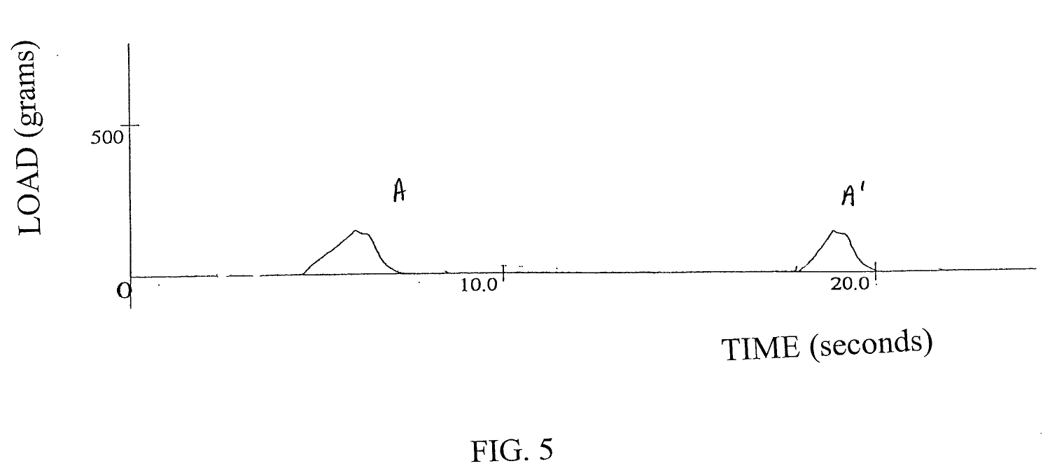

Percent (%) recovery was determined for each sample by calculating the areas of work done for each sample for two cycles. The area of work done for the second cycle divided by the area of work done for the first cycle to provide the percent (%) recovery (i.e., springiness). For example, FIG. 5 illustrates the area of work done for Ex. 7 during the first (A) and second (A′) cycles. Thus,

Area of work done for cycle 1=192.16

Area of work done for cycle 2=136

$$\% recovery = \frac{Area of work done for cycle 2}{Area of work done for cycle 1} = \frac{136}{192.16} = 70.8$$

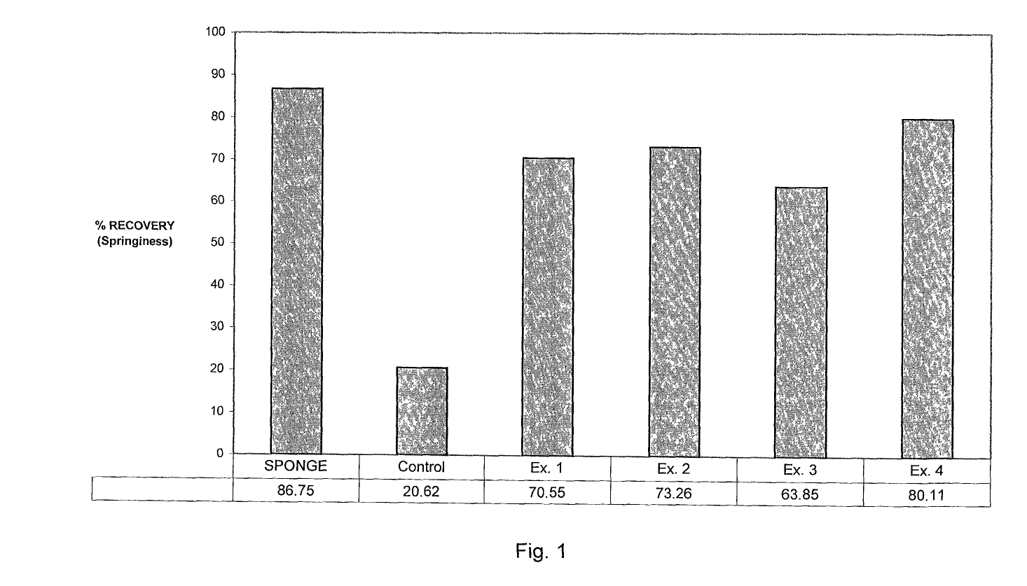

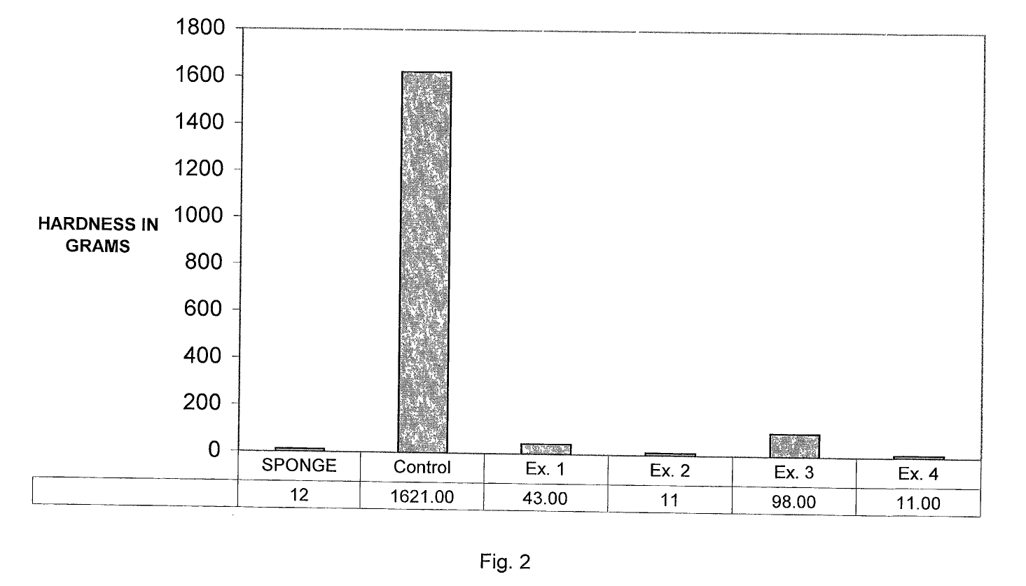

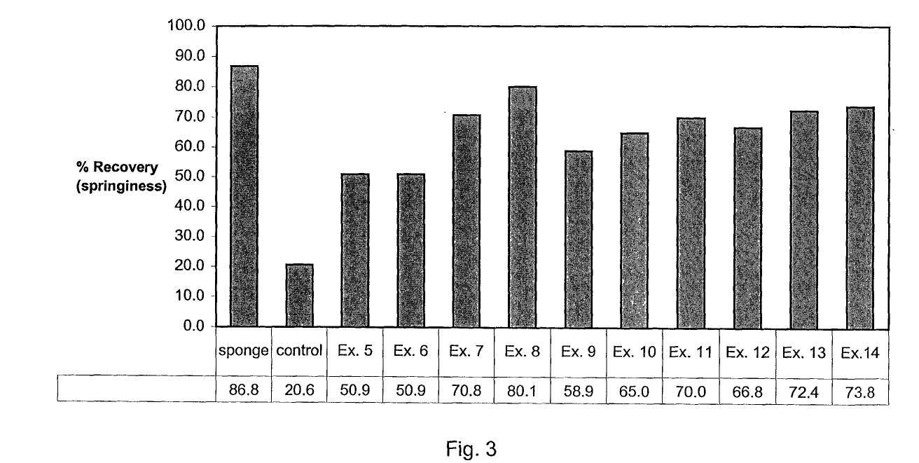

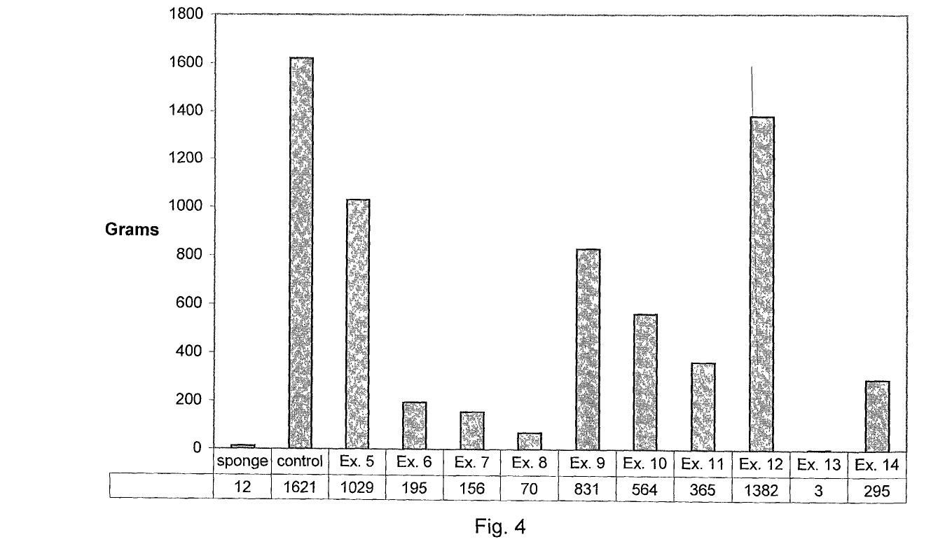

The comparative results for the examples of the present invention relative to the positive (sponge) and negative (prior art pressed powder) controls are illustrated in FIGS. 1 and 3 for springiness and FIGS. 2 and 4 for surface hardness, respectively.

The pressed powder compositions of the present invention exhibited superior binding properties as well as sponginess not heretofore known in prior art cosmetic pressed powders. In other words, the pressed powder compositions of the present invention were able to maintain their shape (i.e., remain a pressed powder) and consistency when pressed into a rigid container just like conventional, hard pressed powders. However, unlike conventional pressed powders, the compositions of the present invention exhibited a novel, heretofore unknown springiness/sponginess feel after being pressed into the container.

Although the most preferred embodiment of the present invention is a pressed powder, the compositions may also be used as loose powders. These loose powder embodiments of the present invention provide a smooth, silky feel on the skin that is superior to powder compositions of the prior art.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A pressed powder cosmetic composition comprising silicone elastomer particulates have a JISA hardness index less than about 45.

2. The pressed powder cosmetic composition of claim 1, wherein the silicone elastomer particulates have a JISA hardness index from about 2 to less than about 45.

3. The pressed powder cosmetic composition of claim 1, wherein the silicone elastomer particulates have a JISA hardness index from about 4 to about 35.

4. The pressed powder cosmetic composition of claim 1, wherein the silicone elastomer particulates have a JISA hardness index from about 20 to about 35.

5. The pressed powder cosmetic composition of claim 1, wherein the composition comprises about 1 wt % to about 25 wt % of the silicone elastomer particulates, based on the total weight of the composition.

6. The pressed powder cosmetic composition of claim 1, wherein the composition comprises about 3 wt % to about 15 wt % of the silicone elastomer particulates, based on the total weight of the composition.

7. The pressed powder cosmetic composition of claim 1, wherein the composition comprises about 3 wt % to about 12 wt % of the silicone elastomer particulates, based on the total weight of the composition.

8. The pressed powder cosmetic composition of claim 1, wherein the pressed powder composition has a hardness value less than about 1500 grams.

9. The pressed powder cosmetic composition of claim 1, further comprising filler selected from group consisting of mineral silicate, starch, kaolin, nylon, zinc oxide, titanium oxide, precipitated calcium carbonate, synthetic polymer powder, or any combinations thereof.

10. The pressed powder cosmetic composition of claim 9, wherein the filler is a mineral silicate or synthetic polymer powder.

11. The pressed powder cosmetic composition of claim 1, further comprising a pigment.

12. The pressed powder cosmetic composition of claim 1, further comprising a synthetic polymer powder.

13. A pressed powder cosmetic composition comprising silicone elastomer particulates, wherein the pressed powder composition has a hardness value less than about 1500 grams.

14. The pressed powder cosmetic composition of claim 13, wherein the pressed powder composition has a hardness value less than about 1100 grams.

15. The pressed powder cosmetic composition of claim 13, wherein the pressed powder composition has a hardness value less than about 900 grams.

16. The pressed powder cosmetic composition of claim 13, wherein the pressed powder composition has a hardness value less than about 600 grams.

17. The pressed powder cosmetic composition of claim 13, wherein the pressed powder composition has a hardness value less than about 300 grams.

18. The pressed powder cosmetic composition of claim 13, wherein the pressed powder composition has a hardness value less than about 100 grams.

19. The pressed powder cosmetic composition of claim 13, wherein the pressed powder composition has a JISA hardness index less than about 45.

20. A pressed powder cosmetic composition comprising comprising silicone elastomer particulates, wherein the pressed powder composition has a percent recovery greater than about 25.

21. The pressed powder cosmetic composition of claim 20, wherein the pressed powder composition has a JISA hardness index less than about 45.

22. The pressed powder cosmetic composition of claim 20, wherein the pressed powder has a percent recovery greater than about 50.

23. The pressed powder cosmetic composition of claim 20, wherein the pressed powder has a percent recovery greater than about 60.

24. The pressed powder cosmetic composition of claim 20, wherein the pressed powder has a percent recovery greater than about 70.

25. The pressed powder cosmetic composition of claim 20, wherein the pressed powder has a percent recovery greater than about 80.

26. A process for making a pressed powder cosmetic composition comprising:
forming a dry powder phase having silicone elastomer particulates;
separately preparing a liquid binder phase;
adding the liquid binder phase to the dry powder phase;
mixing the liquid binder phase and the dry powder phase together to form a blend; and
pressing the blend at a pressure about 800 psi to about 2500 psi.

27. The process of claim 26, wherein the blend is pressed at a pressure about 1,000 psi to about 1,700 psi.

28. A method of imparting springiness to a pressed powder cosmetic composition comprising introducing into the composition silicone elastomer particulate having a JISA hardness index less than about 45.
